(19) United States
(12) Patent Application Publication
(10) Pub. No.: US 2012/0010509 A1
(43) Pub. Date: Jan. 12, 2012

(54) Ultrasonic Vertebral Bone Assessment Apparatus and Method

(75) Inventors: Jonathan J. Kaufman, Brooklyn, NY (US); Gangming Luo, Elmhurst, NY (US)

(73) Assignee: CyberLogic, Inc., New York, NY (US)

(21) Appl. No.: 13/177,892

(22) Filed: Jul. 7, 2011

Related U.S. Application Data
(60) Provisional application No. 61/362,913, filed on Jul. 9, 2010.

(51) Int. Cl. A61B 8/00

(52) U.S. Cl. 600/449

(57) ABSTRACT

A method and apparatus for non-invasive and quantitative assessment of the status of a lumbar vertebral body in a living being for at least one of several quantities (e.g., bone-mineral density, bone mass, etc.) is provided. The method includes the steps of acoustically coupling first and second transducers to nearby skin on opposite sides of a torso of the living being and generating an ultrasound signal and directing the ultrasound signal from the first transducer to the second transducer through the torso. At least a portion of the ultrasound signal passes through the lumbar vertebral body and the second transducer generates an output signal responsive to receipt of the ultrasound signal. The method further includes the step of processing the output signal to obtain an estimate of the at least one quantity.

This application claims priority to U.S. Provisional Patent Application No. 61/362,913 filed Jul. 9, 2010, the entire disclosure of which is incorporated herein by reference.

### BACKGROUND OF THE INVENTION

1. Field of the Invention

The present invention relates generally to apparatus and method for non-invasively and quantitatively evaluating bone tissue in vivo. More specifically, the invention pertains to osteoporosis diagnosis and bone fracture risk assessment, using an ultrasonic device.

2. Discussion of Related Art

In recent years, ultrasound has received a great deal of attention as a new technique for noninvasive assessment of bone, and numerous attempts have been made to use ultrasound energy for evaluating the condition of bone tissue in vivo, and thus for determining a measure of osteoporosis and assessing bone fracture risk.

In particular, Hoop discloses in U.S. Pat. No. 3,847,141 a device to measure bone density as a means for monitoring calcium content of the involved bone. A pair of opposed ultrasonic transducers is applied to opposite sides of a subject's finger, such that recurrent pulses transmitted via one transducer are ‘focused” on the bone, while the receiver response of the other transducer is similarly “focused” to receive pulses that have been transmitted through the bone. The circuitry in Hoop is arranged such that filtered reception of one pulse triggers the next pulse transmission; the filtering is by way of a bandpass filter, passing components of received signals in the 25 kHz to 125 kHz range only; and the observed frequency of retriggering is believed to be proportional to the calcium content of the bone. Thus Hoop is concerned only with what he defines to be transit time for pulses in the indicated band.

Pratt, Jr. deals with establishing, in vivo, the strength of bone in a live being such as a horse. In U.S. Pat. No. 4,361,154, the inventor solves the problem posed by measuring transit time from “launch” to “reception” of pulses of 0.5 MHz and 1.0 MHz through the bone and soft tissue, and from measurement of pulse-echo time, to thereby derive a measurement of transit time through bone alone. A data bank enables the evaluation of the bone condition from the measured transit times. U.S. Pat. No. 4,913,157, also granted to Pratt, Jr., operates on the same general principle of transit time/velocity deduction, using the latter preferred frequency of 2.25 MHz as the base frequency of pulsed “launchings” and a technique of matched filtering/Fourier transform filtering for further analyzing received pulses.

Palmer et al. disclose in U.S. Pat. No. 4,774,959 a bone measurement system deriving the slope of the relation between ultrasonic frequency and attenuation of a sequence of tone signals. Being in the range of 200 kHz to 600 kHz, the signals are applied to one transducer and received by another transducer. The passage of the signals between the two transducers with and without the intervening presence of a heel bone is compared, with the assumption that the frequency/attenuation relation is a straight line, i.e., of constant slope.

U.S. Pat. No. 4,926,870 granted to Brandenburger discloses another in vivo bone analysis system which depends upon measuring transit time for an ultrasonic signal along a desired path through bone. A “canonical” waveform, determined by previous experience to be on the correct path, is used for comparison against received signals for transmission through the patient's bone, while the latter is reoriented until the received signal indicates that the bone is aligned with the desired path. Again, ultrasonic velocity through the patient's bone is assumed to have been determined from measured transit time.

Rossman et al. disclose in U.S. Pat. No. 5,054,490 an ultrasound densitometer for measuring physical properties and integrity of bone, upon determination of a transit time through bone. Alternatively, the Rossman et al. device compares absolute attenuation of specific frequency components of ultrasound signals through the bone with the absolute attenuation of the same frequency components through a medium of known acoustic properties.

Mele et al., disclose in U.S. Pat. No. 5,564,423, and in a subsequent related Patent by Cadossi et al. (U.S. Pat. No. 6,436,042), disclose a device that measures the “amplitude dependent speed of sound” through a bony member in a living body. The method relies on the visual display of the received ultrasound signal, and the selection of a specific portion of the waveform for analysis.

The prior art, exemplified by the above references that have been briefly discussed, proceed on the assumptions that transit time and velocity—as well as the assumed linear slope of attenuation as a function of a set of discrete frequencies—are all-important in assessing bone. These approaches have essentially been ad hoc, with no consistent framework within which to analyze data. Despite the fact that a rich variety of information is obtainable from experiments with ultrasound (including computer simulations as well as in vitro and in vivo experiments) and that a variety of analytic results are available as well, much of the information has not been used and available, and useful aspects of the data have been ignored.

Significant steps forward in this direction have been made by Kaufman et al. (in U.S. Pat. Nos. 5,259,384 and 5,651,363) and by Chiabrera et al. (in U.S. Pat. Nos. 5,785,656 and 5,879,301). In these Patents, an estimate of a “bone transfer function” associated with a given bone is obtained in a statistically optimal fashion, and parametric estimates of the phase and attenuation functions associated with it are determined. The disclosed methods also describe the use of 2D array transducers for obtaining more reproducible estimates of the bone density, architecture, and fracture risk.

Notwithstanding the advances made in the last-mentioned apparatuses and methods, there are still significant improvements needed in order to accurately and precisely assess the bone density, architecture, quality and fracture risk of a subject. While ultrasound techniques have been able to accurately assess the peripheral skeleton, and in spite of all the advantages of ultrasound over x-ray techniques, ultrasound has remained a largely marginal technology. This is because ultrasound has been limited to assessing peripheral anatomic sites (such as the calcaneus and forearm), while standard of medical care specifies assessment at axial (like the hip and spine) sites; heretofore such axial assessment has not been possible with present ultrasound technology.

### SUMMARY OF THE INVENTION

The present invention provides a method and an apparatus for non-invasive and quantitative assessment of the status of a lumbar vertebral body in a living being for at least one of the quantities, bone-mineral density, bone mass, bone mineral content, bone strength, bone fracture risk, bone architecture and bone quality.

A method in accordance with one embodiment of the invention includes the step of acoustically coupling a first transducer and a second transducer to nearby skin on opposite sides of a torso of the living being. The method further includes the step of generating an ultrasound signal and directing the ultrasound signal from the first transducer to the second transducer through the torso. At least a portion of the ultrasound signal passes through the lumbar vertebral body. The second transducer generates an output signal responsive to receipt of the first ultrasound signal. The method further includes the step of processing the output signal to obtain an estimate of the at least one of the quantities, bone-mineral density, bone mass, bone mineral content, bone strength, bone fracture risk, bone architecture and bone quality.

An apparatus in accordance with one embodiment of the invention includes first and second transducers configured to be acoustically coupled to nearby skin on opposite sides of a torso of the living being. The apparatus further includes means for generating an ultrasound signal and directing the ultrasound signal from the first transducer to the second transducer through the torso. At least a portion of the ultrasound signal passes through the lumbar vertebral body and the second transducer generates an output signal responsive to receipt of the ultrasound signal. The apparatus further includes means for processing the output signal to obtain an estimate of the at least one of the quantities, bone-mineral density, bone mass, bone mineral content, bone strength, bone fracture risk, bone architecture and bone quality.

An apparatus and method in accordance with the present invention represents an improvement over conventional systems because the inventive apparatus employs direct assessment of the axial skeleton (e.g., hip, spine) that is of the most concern for fractures as opposed to relying on indirect assessments made from components of the peripheral skeleton (e.g., the calcaneus).

These and other advantages of this invention will become apparent to one skilled in the art from the following detailed description and the accompanying drawings illustrating features of this invention by way of example.

### BRIEF DESCRIPTION OF THE DRAWINGS

FIG. 1 is a diagrammatic view of one embodiment of an apparatus in accordance with the present invention.

FIG. 2 is a slice image through the torso and, in particular, through a region between lowermost rib and the pelvic bone.

FIG. 3 is a dual-emission X-ray absorptiometry (DXA) image from one side of the torso of a living being.

FIG. 4 is a graph illustrating time domain signals propagating along a pathway through soft tissue only and a pathway through soft tissue and vertebral bone.

### DETAILED DESCRIPTION OF EMBODIMENTS OF THE INVENTION

It is accordingly a primary object of this invention to provide an improved method and apparatus for characterizing and determining non-invasively the properties of bone that is part of the axial skeleton. A more particular object of the invention is to provide a method and apparatus for non-invasive and quantitative evaluation of the lumbar spine of a living subject, to make accurate osteoporosis diagnosis and monitoring possible.

Another object is to meet the above object in such a way that the bone tissue evaluation and the osteoporosis diagnosis may be performed with relatively more accuracy, without ionizing radiation, and more efficient means than those previously used.

A further object is to meet the above objects by providing more accurate and precise estimates of bone mass, bone density, bone geometry, bone quality, and bone strength, as compared with means disclosed previously.

A still further object is to meet the above objects by providing methods to obtain new ultrasound parameters which are sensitive to both bone mass, bone geometry, and bone strength.

A yet still further object is to provide an enhanced ability to estimate the fracture risk associated with a given living being.

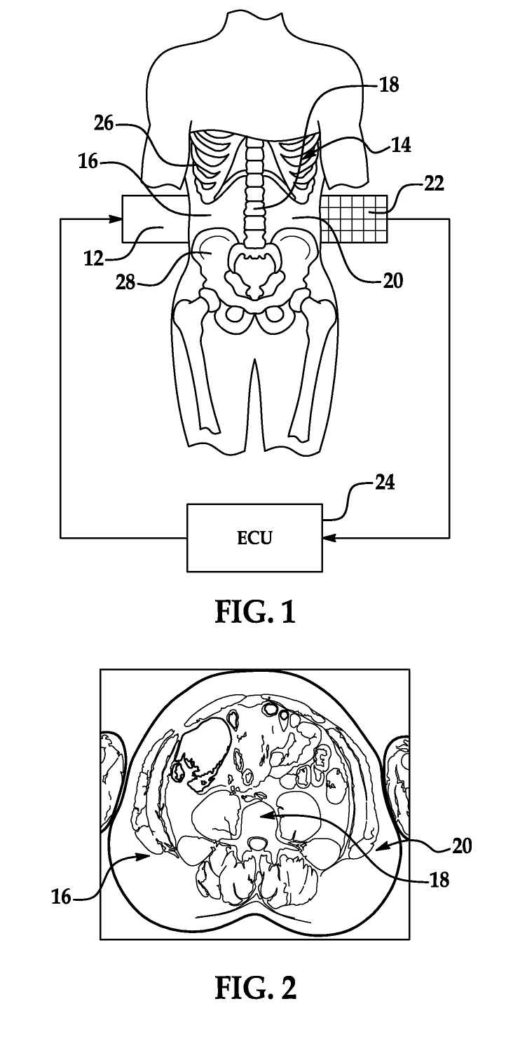

The invention in its presently preferred form was motivated by a key insight of the present inventors. This insight is that there exists in most all individuals an acoustic pathway in the medial-lateral direction that is free of interference producing tissues. This pathway is above the pelvis and below the last (lowest) rib. In this location, the acoustic path consists solely of soft tissue and the lumbar vertebra itself. Therefore, and with reference to FIG. 1, an acoustic wave can be launched by a transmitter 12 (located on one side (medial or lateral) of a torso 14), which will be transmitted through soft tissue 16 towards and through one or more lumbar vertebral bodies 18 (almost always including the third lumbar vertebral body denoted by “L3”), and subsequently through the soft tissue 20 on the other side of the spine, to a receiver transducer 22 placed on the skin on the opposite side (lateral or medial) of the torso 14 of the living person. In the presently preferred embodiment of the invention, a portion of the acoustic wave which has propagated in front of (anterior to) the vertebral body 18 or bodies but not through it is also received by the receiver transducer 22 and used in the computation of the bone properties as disclosed below.

The invention in its presently preferred form of a method of non-invasive and quantitative assessment of the status of the lumbar spine in vivo for one or more of the quantities: bone-mineral density, bone mass, bone mineral content, geometry, strength, quality, and fracture risk, achieves the foregoing objectives by acoustically coupling a pair of transducers 12, 22 to nearby skin surfaces on opposite (i.e., medial and lateral) sides of a torso 14 of a living being; generating an ultrasound signal from a single element transmitter transducer 12 of one of said pair of transducers 12, 22, said ultrasound signal being a finite-duration signal repeated substantially in a range from 1 to 5000 Hz (for averaging) and consisting of plural frequencies spaced in an ultrasonic spectral region up to about 5 MHz; and directing each ultrasound signal from said single element transmitter transducer 12 through the torso 14 where it is received by the other transducer 22, said the other transducer 22 of the said pair of transducers 12, 22 being a rectangular 2D array transducer, and thereby producing a set of received signals associated with the elements of the 2D receiver array; and processing said set of received signals to obtain at least one of a net time delay (NTD) parameter and a mean time duration (MTD) parameter, and further processing the at least one of said NTD parameter and said MTD parameter to obtain an estimate of the one or more said quantities.

The step of further processing may be performed with the use of one or more of a plurality of associated parameters: age, sex, fracture history, bone mineral density as measured by x-ray absorptiometry at a given anatomical site, cigarette smoking history, height, and weight that is specific to an individual subject. The step of further processing may be performed with the use of multivariate linear and nonlinear regressions, a statistical hypothesis testing algorithm, and may include a neural network configured to generate an estimate of the one or more of the quantities from the parameters and from the associated parameters specific for an individual patient.

In its presently preferred apparatus form, the invention comprises transducer means including a pair of ultrasonic transducers 12, 22 adapted for acoustic coupling to nearby skin surfaces on opposite sides of a torso 14 of a living being; and for transmission through an acoustic propagation path which includes a lumbar spine of a living body; a generator means 24 for connecting to a transmission transducer 12 of the pair to generate an ultrasound waveform, this waveform being a finite-duration signal consisting of plural frequencies spaced in the ultrasonic spectral region to approximately 5 MHz and being repeated substantially in the range from 1 Hz to 5000 Hz; and a signal-processing means 24 that are connected for response to the signals received by a receiving transducer 22 of the pair, said receiving transducer 22 being a 2D array transducer, and comprises means to provide analog-to-digital sampling and signal processing of said received signals, to thereby produce corresponding parameters and means for performing further analysis of the parameters resulting in estimates of bone properties. The generating means may comprise an arbitrary-function generator card installed in an electronic control unit 24. The card may suitably be a waveform synthesizer, a product of PC Instruments, Inc., Lawrence, Kans., identified by PC Instruments part No. PCI-341. This card is relied upon to generate an excitation signal which is periodically supplied to the launch transducer 12, via a power amplifier (not shown). The power amplifier is suitably the Model No. 240L, an RF power-amplifier product of EIN, Inc., Rochester, N.Y.. This amplifier provides a 50 dB gain, over the range 20 kHz to 10 MHZ. In addition to power amplifier, the excitation signal must pass through a switching network when transducer 12 is a multi-element, linear- or two-dimensional array transducer. ECU 24 may comprise a programmable microprocessor or microcontroller or may comprise an application specific integrated circuit (ASIC). ECU 24 may include a central processing unit (CPU) for processing the received signals output by transducer 22 and an input/output (I/O) interface through which ECU 24 may receive a plurality of signals including signals generated by transducer 22 and generate a plurality of signals including those used to control transducer 12. ECU 24 may be configured with appropriate programming instructions or code (i.e., software) to perform several steps in the inventive method.

In the presently preferred embodiment of the invention, an ultrasound signal is generated and sent from the source transducer 12, through the torso 14 and to the receiving 2D array receiver transducer 22 where it is measured and processed. The array receiver 22 is used to obtain a set of received (or output) signals. Each signal in the set of received or output signals is associated with an element of the receiver array. In the presently preferred embodiment, two (2) parameters are computed from the set of received signals, the net time delay (NTD) and the mean time duration (MTD) parameters, respectively. The NTD is the difference between the time delay of an ultrasound signal which was propagated through a soft tissue only pathway and the time delay of an ultrasound signal which has propagated not only through soft tissue 16, 20 but also through a relatively central portion of a vertebral body 18 within the living being. This is represented mathematically as NTD=τₛ−τ_{b}, where τₛ is the time delay associated with a signal which had propagated through a soft tissue only path and τ_{b} is the time delay associated with a signal which had propagated through the torso 14 in a path that besides soft tissues 16, 20 on both sides of the spine also contains propagation through a central portion of a vertebral body 18. The MTD is the time span of a given portion of the received signals, and is generally inversely related to the mean frequency of the signal. In the presently preferred embodiment of the invention, both the NTD and MTD are computed using only the first (half) cycle of the received signal, as the present inventors have found that the later portion of the signal is often corrupted by components having little to do with the condition of the bone tissue per se, for example multiple reflections as well as multiple propagation pathways. Further NTD and MTD are evaluated using a highly robust (from a statistical perspective) approach, which is described in the U.S. Published Patent Application 20050197576, filed Sep. 8, 2005, all of which is incorporated by reference hereinto.

In the presently preferred embodiment of the invention, the bone mass as represented by either bone mineral density (BMD) or bone mineral content (BMC) of the lumbar vertebral body 18 is evaluated according to a linear regression between NTD and BMD, i.e., either by BMD=a1·NTD+b1, or by BMC=a2·NTD+b2. The fracture risk associated with the living person is provided by a feedforward neural network whose inputs are the ultrasound parameters NTD and MTD, and the associated parameters age, sex, weight, height and history of fracture. The output of the neural network is the probability of fracture, a number between 0 and 1.

In the presently preferred embodiment of the invention, the source transducer 12 is rectangular (7.5 cm (I-F)×15 cm (A-P)), single element, of nominal center frequency of 1 MH and 70% (6 dB) bandwidth. Such a transducer can be fabricated by a number of companies; our presently preferred embodiment is available from Valpey Fisher Corporation, 75 South Street, Hopkinton, Mass. The receiver array 22 is identical in size and frequency and bandwidth to the source; however it is composed of 72 1.25 cm×1.25 cm square elements. It should be appreciated that the signals associated with the receiver array, i.e., the set of received signals, are input to a suitably adapted electrical multiplexer, in order to suitably digitize and store and process the set of received signals in order to obtain the ultrasound parameters. Such methods and apparatuses for multiplexing and digitizing such signals are well known in the art; for example see the U.S. Pat. Nos. 5,879,301, 5,785,656, 5,651,363, and 5,259,384, as well as the U.S. Patent Applications Nos. 20080194952, 20080146927, and 20050197576, all of which are incorporated by reference hereinto.

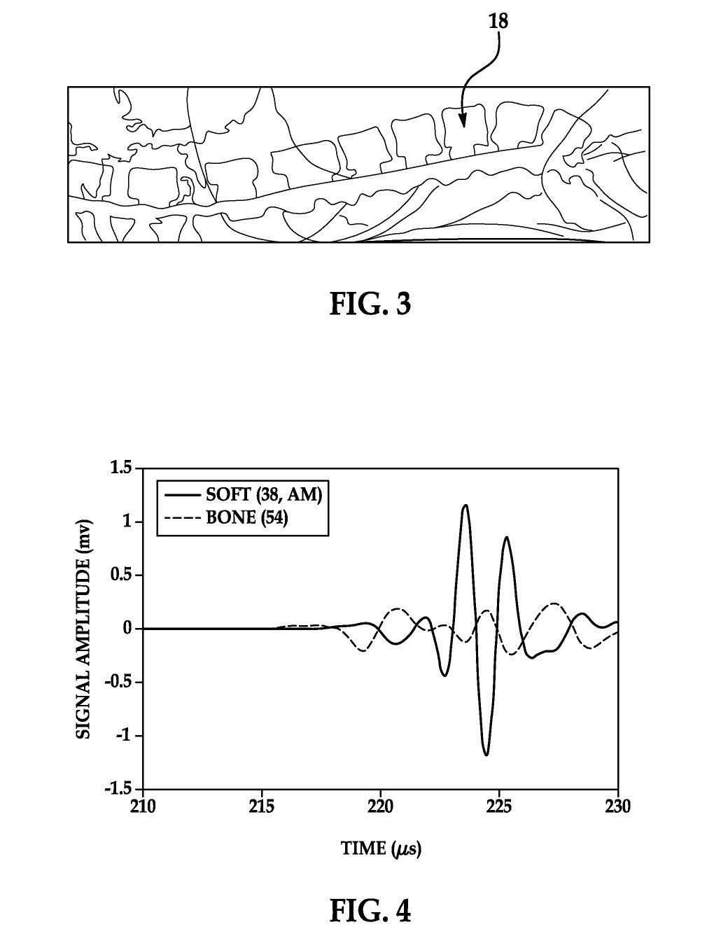

As noted above, a key insight of the present inventors was the realization that an unobstructed acoustic path exists between the source and receiver transducers 12, 22. In the presently preferred embodiment vertebra L3 is assessed because it is the lumbar vertebral body 18 with the most direct access. In some individuals, L2 (lumbar vertebra 2) would have a rib 26 interfering with the acoustic propagation pathway, while with L4 (lumbar vertebra 4) there is often interference from the pelvis 28. This is described in the book by S. L. Bonnick “Bone densitometry in Clinical Practice”, 2^{nd} Edition, published by Humana Press of Totowa, N.J., in 2004, all of which is incorporated by reference hereinto. It may also be appreciated by looking at the image shown in FIG. 2 accessed from the Visual Human Data Set <http://www.nlm.nih.gov.research/visible/visiblehuman.html] that demonstrates this clear acoustic path, as well as FIG. 3 which shows this as well in a lateral DXA scan of an individual. As may be seen, only skin, fat, and muscle, besides the vertebral body itself, are present in a pathway between the medial and lateral (skin) surfaces of the torso 14 of an individual, where the pathway is defined to be below the lowest rib 26 and above the pelvis 28.

In an alternative embodiment of the invention, an array transducer receiver is not used. Instead, two single element transducers (Panametrics Videoscan 1 MHz ¾ inch diameter) serve as receiver and source, respectively. A person lies in a supine position (e.g., on a platform supported about 40 cm above the floor by legs) between the two transducers. Two small water baths on the left and right sides, respectively are placed against the skin with coupling gel and allow the transducers to be mechanically scanned. The rectangular face of the water baths are placed between the lowest rib and the upper part of the pelvis. A y-z stepper-motor system attached to the platform (here y being associated with the inferior-superior (I-S) direction, and z being associated with the anterior-posterior (A-P) direction) allows movement of the transducers and scanning of a rectangular region approximately 7.5 cm (I-F) by 15 cm (A-P) in 5 mm steps.

Through-transmission ultrasound data was obtained from this alternative embodiment and the received waveforms for each spatial position were digitized using a LeCroy 9450 digital oscilloscope and downloaded to a PC. The data showed signals which had a strong soft-tissue component, and channels which were associated with signals that had propagated through the central body of the vertebra. As may be seen in FIG. 4, the soft tissue signal (“Soft (38, AM)”) with a propagation pathway through soft tissue only has a center frequency of about 555 kHz, and the bone signal (“Bone (54)”) with a propagation pathway through soft tissue as well as the body of a vertebra has a center frequency of about 333 kHz, indicating the low-pass filtering effect of the trabecular bone within the vertebral body (and also suggesting that a key parameter associated with the scattering of ultrasound within trabecular bone (i.e., mean time duration can be useful for assessing bone characteristics. The soft tissue signal is from a channel that is associated with a position that is anterior to the vertebral body.

In a further embodiment of the invention, an ultrasound imaging machine is utilized to determine which lumber vertebra is being ultrasonically interrogated. While in the presently preferred embodiment of the invention as disclosed supra, lumbar vertebral body L3 is interrogated in the vast majority of instances, because of inter-subject variations in anatomy, it is possible that vertebral body L2 or L4 may in fact be measured. In order to ensure greater accuracy in terms of which vertebral body 18 is interrogated, in this further embodiment of the invention ultrasound imaging is utilized in order to identify the specific lumbar vertebra for which the bone mass and other bone-features are being determined. In yet another further embodiment of the invention, an x-ray of the living being is used to determine which lumbar vertebra is ultrasonically assessed. The information gained in the two further embodiments of the invention, that is using ultrasound or x-ray images to accurately identify a vertebral body, may be used to specify the bone density of a specific vertebral body (e.g., L2, L3, or L4), or alternatively could be used to provide an estimate of the bone density of L3. For example, if the imaging shows that L3 has been ultrasonically interrogated, the value of bone density is used. On the other hand if L4 has been interrogated, then a percentage of L4 bone density is used to estimate L3 bone density. Similarly, if L2 has been ultrasonically interrogated, then a percentage of the estimated bone density of L2 is used to estimate L3 bone density. In these further embodiments of the invention, L3 bone density=0.95 of L4 bone density and 1.07 of L2 bone density.

While several embodiments of the present invention have been disclosed hereinabove, it is to be understood that these embodiments are given by example only and not in a limiting sense. Those skilled in the art may make various modifications and additions to the preferred embodiments chosen to illustrate the invention without departing from the spirit and scope of the present contribution to the art. Accordingly, it is to be realized that the patent protection sought and to be afforded hereby shall be deemed to extend to the subject matter claimed and all equivalence thereof fairly within the scope of the invention.

I claim:

1. A method of non-invasive and quantitative assessment of the status of a lumbar vertebral body in a living being for at least one of the quantities, bone-mineral density, bone mass, bone mineral content, bone strength, bone fracture risk, bone architecture and bone quality, comprising the steps of:
acoustically coupling a first transducer and a second transducer to nearby skin on opposite sides of a torso of said living being;
generating a first ultrasound signal and directing said first ultrasound signal from said first transducer to said second transducer through said torso, at least a first portion of said first ultrasound signal passing through said lumbar vertebral body, said second transducer generating a first output signal responsive to receipt of said first ultrasound signal; and,
processing said first output signal to obtain an estimate of said at least one of the quantities, bone-mineral density, bone mass, bone mineral content, bone strength, bone fracture risk, bone architecture and bone quality.

2. The method of claim 1 wherein said vertebral body is an L3 vertebra.

3. The method of claim 1 wherein said second transducer is an array transducer.

4. The method of claim 1 wherein said first ultrasound signal traverses a path between a lowermost rib in said living being and above a pelvis in said living being.

5. The method of claim 1 wherein said processing step includes the substep of obtaining a net time delay parameter associated with said first ultrasound signal.

6. The method of claim 5 wherein said net time delay parameter is determined responsive to a comparison between a time for said first portion of said first ultrasound signal to travel along a first path between said first and second transducers through said lumbar vertebral body and a time for a second portion of said ultrasound signal to travel along a second path between said first and second transducers through soft tissue.

7. The method of claim 1 wherein said processing step includes the substep of obtaining a mean time duration parameter associated with said first ultrasound signal.

8. The method of claim 7 wherein said mean time duration parameter is determined responsive to a time span of a predetermined portion of said first ultrasound signal.

9. The method of claim 1, further comprising the steps of:
generating a second ultrasound signal and directing said second ultrasound signal from said first transducer to said second transducer through said torso, but not through said lumbar vertebral body, said second transducer generating a second output signal responsive to receipt of said second ultrasound signal; and,
processing said second output signal to obtain said estimate of said at least one of the quantities, bone-mineral density, bone mass, bone mineral content, bone strength , bone fracture risk, bone architecture and bone quality.

10. The method of claim 9 wherein said processing step includes the substep of obtaining a net time delay parameter associated with said first and second ultrasound signals, said net time delay parameter determined responsive to a comparison between a time for said first ultrasound signal to travel along a first path between said first and second transducers through said lumbar vertebral body and a time for said second ultrasound signal to travel along a second path between said first and second transducers through soft tissue.

11. An apparatus for non-invasive and quantitative assessment of the status of a lumbar vertebral body in a living being for at least one of the quantities, bone-mineral density, bone mass, bone mineral content, bone strength, bone fracture risk, bone architecture and bone quality, comprising:
first and second transducers configured to be acoustically coupled to nearby skin on opposite sides of a torso of said living being;
means for generating a first ultrasound signal and directing said first ultrasound signal from said first transducer to said second transducer through said torso, at least a first portion of said first ultrasound signal passing through said lumbar vertebral body, said second transducer generating a first output signal responsive to receipt of said first ultrasound signal; and,
means for processing said first output signal to obtain an estimate of said at least one of the quantities, bone-mineral density, bone mass, bone mineral content, bone strength, bone fracture risk, bone architecture and bone quality.

12. The apparatus of claim 11 wherein said vertebral body is an L3 vertebra.

13. The apparatus of claim 11 wherein said second transducer is an array transducer.

14. The apparatus of claim 11 wherein said first ultrasound signal traverses a path between a lowermost rib in said living being and above a pelvis in said living being.

15. The apparatus of claim 1 wherein said processing means includes means for obtaining a net time delay parameter associated with said first ultrasound signal.

16. The apparatus of claim 15 wherein said net time delay parameter is determined responsive to a comparison between a time for said first portion of said first ultrasound signal to travel along a first path between said first and second transducers through said lumbar vertebral body and a time for a second portion of said ultrasound signal to travel along a second path between said first and second transducers through soft tissue.

17. The apparatus of claim 11 wherein said processing means includes means for obtaining a mean time duration parameter associated with said first ultrasound signal.

18. The apparatus of claim 17 wherein said mean time duration parameter is determined responsive to a time span of a predetermined portion of said first ultrasound signal.

19. The apparatus of claim 1, further comprising:
means for generating a second ultrasound signal and directing said second ultrasound signal from said first transducer to said second transducer through said torso, but not through said lumbar vertebral body, said second transducer generating a second output signal responsive to receipt of said second ultrasound signal; and,
means for processing said second output signal to obtain said estimate of said at least one of the quantities, bone-mineral density, bone mass, bone mineral content, bone strength, bone fracture risk, bone architecture and bone quality.

20. The apparatus of claim 19 wherein said processing means includes means for obtaining a net time delay parameter associated with said first and second ultrasound signals, said net time delay parameter determined responsive to a comparison between a time for said first ultrasound signal to travel along a first path between said first and second transducers through said lumbar vertebral body and a time for said second ultrasound signal to travel along a second path between said first and second transducers through soft tissue.
